(19) United States
(12) Patent Application Publication
(10) Pub. No.: US 2026/0131104 A1
(43) Pub. Date: May 14, 2026

(54) AIR-INJECTION TYPE GARMENT HAVING PIEZOELECTRIC ELEMENT DESIGNED TO MEASURE BALLISTO CARDIOGRAM

(71) Applicant: DOLBOMDREAM CO. LTD., Daejeon (KR)

(72) Inventors: Jihun Kim, Goyang-si (KR); Seokhyun Kim, Daejeon (KR)

(73) Assignee: DOLBOMDREAM CO. LTD., Daejeon (KR)

(21) Appl. No.: 19/412,922

(22) Filed: Dec. 9, 2025

Related U.S. Application Data
(63) Continuation-in-part of application No. PCT/KR2024/005758, filed on Apr. 29, 2024.

(30) Foreign Application Priority Data
Jun. 16, 2023 (KR) 10-2023-0077740
Jun. 16, 2023 (KR) 10-2024-0008276

(51) Int. Cl. A61M 21/02; A61H 9/00; A61M 21/00

(52) U.S. Cl. CPC A61M 21/02; A61H 9/005; A61H 2201/165; A61H 2201/5012; A61H 2201/5071; A61H 2230/045; A61M 2021/0022; A61M 2205/0294; A61M 2205/3344; A61M 2205/3584; A61M 2230/04; A61M 2230/63

(57) ABSTRACT

A wearable device includes: an outer shell including a front portion and a back portion, the outer shell including a first surface and a second surface; an air tube disposed between the first surface and the second surface; an air inlet provided at one end of the air tube; a driving unit configured to inject air into or discharge air from the air tube through the air inlet; a silicone tube extending from the driving unit and connected to the air inlet, a first end of the silicone tube forming an opening and being connected to the air inlet, and a second end of the silicone tube being formed in a sealed structure; a sensor module including a piezoelectric element configured to sense pressure; and a processor disposed inside the silicone tube and operatively coupled to the driving unit and the sensor module.

### CROSS REFERENCE TO PRIOR APPLICATIONS

This application is a continuation application of PCT International Patent Application No. PCT/KR2024/005758 filed on Apr. 29, 2024, which claims priority to Korean Patent Application Nos. 10-2023-0077740 filed on Jun. 16, 2023, and 10-2024-0008276 filed on Jun. 16, 2023 which are all hereby incorporated by reference in their entirety.

### BACKGROUND

The present disclosure relates to an inflatable garment configured to manage a user's psychological state based on biometric information, and more particularly, to an inflatable garment including a sensor module having a piezoelectric element configured to measure ballistic heart vibrations of the user.

In recent years, the importance of managing and stabilizing a user's psychological state or stress level has continued to increase, creating a growing need for methods and devices capable of alleviating anxiety and stress. In this context, various approaches have been proposed in which a wearable device equipped with a biometric sensor acquires biometric information of a user and determines or estimates the user's psychological state or stress based on the acquired biometric information.

Conventional techniques for determining or estimating a psychological condition or stress level typically rely on biometric sensors designed to directly contact the user's body. Although such contact-type biometric sensors are capable of acquiring biometric information in real time, they often cause discomfort, foreign-body sensation, or a feeling of restriction due to the physical contact with the user's body.

To address this issue, non-contact biometric sensors have been introduced as an alternative capable of acquiring biometric information without requiring direct contact with the user's skin. However, even with non-contact biometric sensing, motion-induced noise generated by the user's body movement tends to be more pronounced compared to contact-type sensing, thereby degrading the accuracy of the biometric information.

Accordingly, there is a need for an air-inflatable compression garment that incorporates a method capable of acquiring biometric information without contacting the user's body—thereby eliminating discomfort—while also minimizing noise caused by the user's motion so that more accurate biometric information can be obtained.

### SUMMARY

To determine or estimate a user's psychological state or stress level, it is essential to acquire accurate biometric information of the user. In particular, minimizing noise in the biometric information that forms the basis of such determinations is critical for achieving higher precision. Therefore, a method is required that enables the acquisition of biometric information in real time while minimizing noise.

Although contact-type sensors have conventionally been used to acquire biometric information in real time, such sensors inevitably create discomfort due to their physical contact with the user. Non-contact biometric sensing has been proposed as an alternative; however, biometric data obtained through non-contact sensing generally contains more noise caused by the user's body movements than data acquired through contact-type sensing.

Thus, there is a demand for an air-inflatable garment incorporating a non-contact biometric sensing mechanism that reduces user discomfort while still minimizing motion-induced noise and enabling accurate acquisition of the user's biometric information.

According to one embodiment, a wearable device for caring for a psychological state of a user based on biometric information of the user may include an outer shell having a front portion positioned at a chest region of the user and a rear portion positioned at a back region of the user. The outer shell may include a first surface facing a body of the user and a second surface facing away from the body of the user.

The wearable device may include an air tube disposed between the first surface and the second surface, and an air inlet provided at one end of the air tube such that air is injected into or discharged from the air tube. The wearable device may further include a driving unit configured to inject air into or discharge air from the air tube through the air inlet; a silicone tube extending from the driving unit and connected to the air inlet; a sensor module disposed inside the silicone tube and including a piezoelectric element configured to sense pressure; and a processor disposed inside the silicone tube and operatively coupled to the driving unit and the sensor module.

According to one embodiment, the processor is configured to acquire air-pressure data representing an air pressure inside the air tube through the piezoelectric element of the sensor module, determine a state of the user based on the air-pressure data, the state of the user including at least one of a psychological state, a stress level, or a type of physical symptom, and control the driving unit to inject air into the air tube based on the determined state of the user.

According to one embodiment of the present disclosure, an air-inflatable garment equipped with a biometric module including a piezoelectric element enables the acquisition of biometric information necessary for determining and caring for a psychological state or stress level of a user without direct contact with the user's skin and without restricting the user.

In addition, by measuring air-pressure data inside a sealed silicone tube using the piezoelectric element, the wearable device can derive cardio-mechanical information of the user and obtain biometric information based thereon. As a result, the biometric information includes significantly reduced noise compared to conventional non-contact sensing methods. This allows the psychological state or stress level of the user to be determined with higher precision, and air-pressure compression may be applied to the user based on the determined state to effectively reduce anxiety or stress.

Various additional advantages that are directly or indirectly derived from the present disclosure may also be provided.

### BRIEF DESCRIPTIONS OF THE DRAWINGS

FIG. 1 illustrates a system for caring for a psychological state of a user using a wearable device according to an embodiment.

FIG. 2 illustrates a wearable device having a sensor module configured to measure cardio-mechanical activity according to an embodiment.

FIG. 3 illustrates an internal configuration of the sensor module configured to measure cardio-mechanical activity according to an embodiment.

FIG. 4 illustrates a block configuration of the sensor module configured to measure cardio-mechanical activity according to an embodiment.

FIG. 5 illustrates a sectional view of a fabric structure of the wearable device according to an embodiment.

FIG. 6 illustrates a conceptual diagram of a technique for acquiring biometric information of a user using the sensor module provided in the wearable device according to an embodiment.

FIG. 7 illustrates a conceptual diagram of a technique for correcting biometric information using movement information of the user according to an embodiment.

FIG. 8 illustrates a flowchart of a method for caring for a psychological state using biometric information according to an embodiment.

FIG. 9 illustrates a flowchart of an operation between a wearable device and a server for caring for a psychological state using biometric information according to an embodiment.

In the drawings, identical or similar reference numerals may denote identical or similar components.

### DETAILED DESCRIPTION OF THE INVENTION

Various embodiments of the present invention will now be described with reference to the accompanying drawings. However, the embodiments are not intended to limit the present invention to specific forms, and the present invention should be understood as encompassing various modifications, equivalents, and/or alternatives.

The embodiments described below are provided so that those skilled in the art may implement the present invention without undue experimentation, with reference to the drawings. The present invention may be embodied in many different forms and should not be construed as limited to the embodiments set forth herein. In the drawings, certain elements unrelated to the description of the invention may be omitted for clarity, and like reference numerals designate like elements throughout the specification.

Throughout the specification, when a part “includes” an element, unless otherwise described, it should be understood as meaning that other elements are not excluded and may further be included. In addition, the terms “unit,” “module,” “device,” and the like, described herein, refer to components that perform at least one function or operation, and may be implemented as hardware, software, or a combination thereof.

Throughout the specification, when a part is described as being “connected” to another part, it should be understood to include not only direct physical or electrical connection but also indirect electrical connection through one or more intermediate components. Furthermore, when a part “includes” an element, unless otherwise described, it should be understood that other elements may additionally be included, and that the presence or addition of one or more features, numbers, steps, operations, components, or combinations thereof is not excluded in advance.

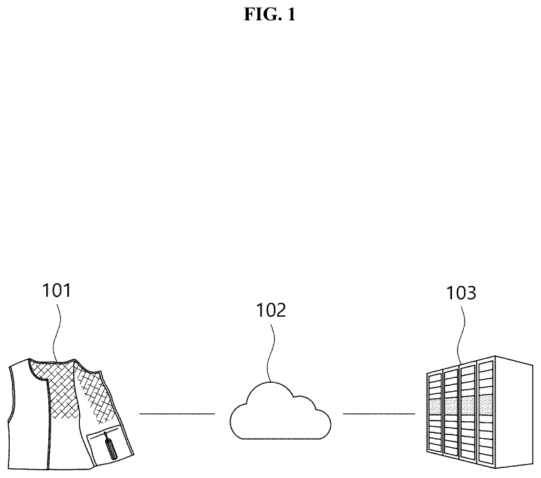

FIG. 1 illustrates a system for caring for a psychological state of a user using a wearable device 101 according to an embodiment.

Referring to FIG. 1, the system for caring for a psychological state of a user may include a wearable device 101, a network 102, and a server 103. However, the system is not limited to the components illustrated in FIG. 1, and certain components may be omitted or additional components may be added. For example, the system may further include a user terminal (e.g., a smartphone).

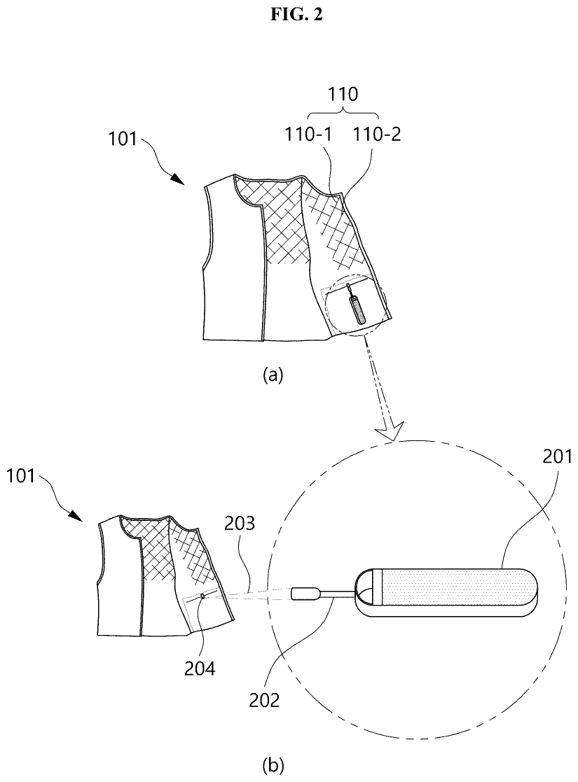

According to one embodiment, the wearable device 101 may be an air-inflatable garment including an air tube (e.g., the air tube 110 in FIG. 2), an air inlet (e.g., the air inlet 204 in FIG. 2), and a driving unit for providing deep touch pressure (DTP) to a user wearing the wearable device 101. For example, the wearable device 101 may be designed to provide deep touch pressure to a user who feels psychological anxiety or elevated stress, thereby relieving the anxiety and reducing the stress level. Deep touch pressure refers to pressure applied to the user's body to stimulate the parasympathetic nervous system, producing a sensation similar to being hugged and thereby promoting psychological stability. The user may include a person requiring psychological comfort or stress relief, such as a child or adult with developmental disabilities. However, the user is not limited to these examples and may include infants, children, adolescents, individuals with disabilities, or elderly individuals.

According to one embodiment, the driving unit may function as an air pump and may inject air into the air tube (e.g., the air tube 110) through the air inlet (e.g., the air inlet 204), or discharge air from the air tube. For example, when it is determined that a user wearing the wearable device 101 is experiencing anxiety or stress, the driving unit may inject air into the air tube through the air inlet. After air is injected and it is determined that the user's anxiety or stress has been alleviated, the driving unit may discharge air from the air tube. Determination of whether the user is experiencing anxiety or stress may be based on biometric information acquired through the sensor module provided in the wearable device 101 (e.g., sensor module 201). For example, if one or more values included in the biometric information deviate from a preset value or preset range, the wearable device 101 may determine that the user is feeling anxious or stressed.

According to one embodiment, the wearable device 101 may further include a communication module configured to perform data communication with an external electronic device. For example, the wearable device 101 may communicate with the server 103 through the network 102. The network 102 may represent wireless communication and may include a mobile hotspot and/or Wi-Fi.

According to one embodiment, the wearable device 101 may transmit biometric information acquired through the sensor module (e.g., sensor module 201), such as cardio-mechanical information, heart rate variability information, or respiratory information, to the server 103 through the network 102. The wearable device 101 may also receive, from the server 103, an artificial intelligence model updated by the server 103.

According to one embodiment, the server 103 may perform data communication with the wearable device 101 and may operate an artificial intelligence model configured to generate control values for controlling the driving unit of the wearable device 101. For example, the server 103 may train an artificial intelligence model using training data and may obtain output data by inputting data into the trained model.

According to one embodiment, when data communication between the wearable device 101 and the server 103 is established, the server 103 may receive biometric information of the user from the wearable device 101. When such communication is established, the server 103 may also transmit an updated artificial intelligence model to the wearable device 101.

According to one embodiment, although not illustrated in FIG. 1, the wearable device 101 may communicate with a user terminal (e.g., a smartphone) through the network 102. For example, the wearable device 101 may transmit biometric information acquired through the sensor module (e.g., sensor module 201) to the user terminal. The user terminal may transmit command data to the wearable device 101 through the network 102 to control the wearable device 101.

FIG. 2 illustrates a wearable device 101 including a sensor module 201 configured to measure ballistocardiogram (BCG) signals according to an embodiment.

Referring to FIG. 2, the wearable device 101 may include a sensor module 201 including a piezoelectric element capable of measuring BCG signals, an air tube 110 including a first adhesive portion 110-1 of a first type and a second adhesive portion 110-2 of a second type, an air inlet 204, and a driving unit (not shown).

According to one embodiment, although not illustrated in FIG. 2, the wearable device 101 is not limited to including only the sensor module that measures BCG using a piezoelectric element, and may further include various biometric sensors configured to measure different types of biometric information. For example, the biometric sensors may include a sensor for measuring electrodermal activity (EDA), a sensor for measuring photoplethysmography (PPG), a sensor for measuring blood volume pulse (BVP), or a sensor for measuring thermal information (e.g., body temperature).

According to one embodiment, the wearable device 101 is described hereinafter as a vest-type garment including the air tube 110. However, the wearable device 101 is not limited to a vest-type garment and may include one or more garments capable of applying deep touch pressure to the user. According to one embodiment, when worn by a user, the wearable device 101 may include a front portion contacting a chest region of the user and a rear portion contacting a back region of the user.

According to one embodiment, in order for air injected through the driving unit to evenly compress the chest region of the user wearing the wearable device 101, the wearable device 101 may include the first adhesive portion 110-1 of a first type and the second adhesive portion 110-2 of a second type. The first adhesive portion 110-1 may be formed as circular or point-type bonding areas, while the second adhesive portion 110-2 may be formed as line-type bonding areas having a predetermined length. According to one embodiment, the air tube 110 included in the wearable device 101 may form an air passage through alternate arrangement of the first adhesive portion 110-1 and the second adhesive portion 110-2. According to one embodiment, the alternating arrangement of the first adhesive portion 110-1 and the second adhesive portion 110-2 may form an adhesive line, and a plurality of such adhesive lines may be arranged laterally across the front and rear portions of the wearable device 101. The second adhesive portion 110-2 may be formed as a straight line or as a curve having a predetermined curvature.

According to one embodiment, the sensor module 201 may include a piezoelectric element disposed inside a housing and a silicone tube 202 configured to seal the piezoelectric element. For example, the silicone tube 202 may enclose the piezoelectric element or a PCB (printed circuit board) on which the piezoelectric element is mounted, such that the PCB is entirely sealed inside the silicone tube.

According to one embodiment, the air tube 110 provided in the wearable device 101 may be configured as a sealed structure except for the air inlet 204 through which air is injected or discharged. An end of the silicone tube 202 extending from the sensor module 201 may be connected (203) to the air inlet 204 of the wearable device 101. The silicone tube 202 extending from the sensor module 201 may be configured as a sealed structure except for its end connected to the air inlet 204. Accordingly, by connecting the end of the silicone tube 202 to the air inlet 204, the pressure of the sealed air in the air tube 110 and the silicone tube 202 may be applied to the piezoelectric element.

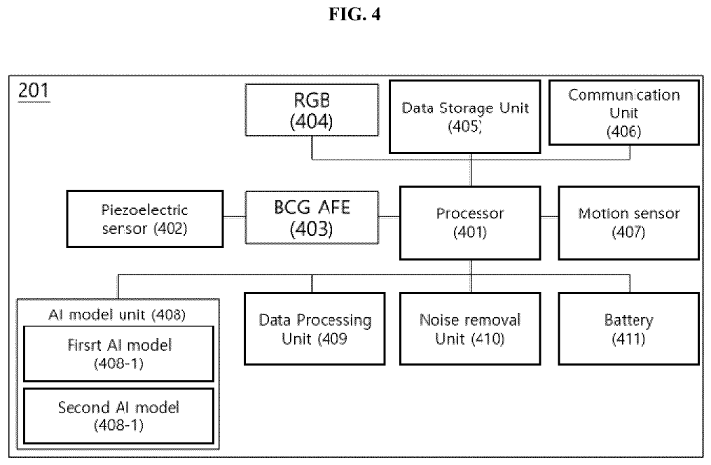

The piezoelectric element may detect air-pressure changes in the wearable device 101 caused by cardiac activity and/or respiration of a user wearing the wearable device 101. A processor of the wearable device 101 (e.g., processor 401 in FIG. 4) may acquire BCG data based on data detected by the piezoelectric element, such as air-pressure variation data.

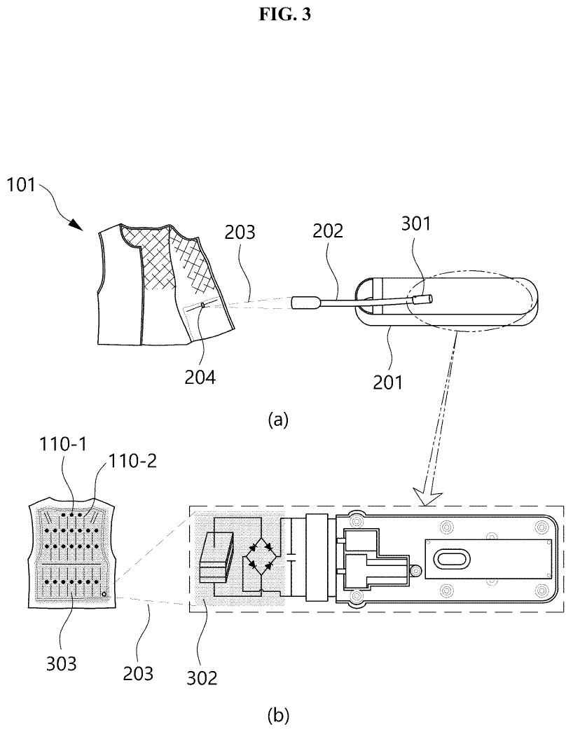

FIG. 3 illustrates an internal configuration of the sensor module 201 configured to measure ballistocardiogram (BCG) signals according to an embodiment.

Referring to FIG. 3, the sensor module 201 may include a piezoelectric element, and a silicone tube 202 may be disposed to seal 302 (or encapsulate) the piezoelectric element. The wearable device 101 may include an air tube 110 that forms a sealed space 303 in which air is injected across both the front portion and the rear portion of the wearable device 101. The air tube 110 may include a first type of air-adhesive portion 110-1 and a second type of air-adhesive portion 110-2.

According to one embodiment, when the silicone tube 202 of the sensor module 201 is connected to the air inlet 204 of the wearable device 101, the piezoelectric element of the sensor module 201 may detect changes in air pressure inside the air tube 110 based on cardiac activity and/or respiration of a user wearing the wearable device 101.

According to one embodiment, a processor of the sensor module 201 (e.g., the processor 401 in FIG. 4) may amplify a small output voltage generated by the piezoelectric element using an amplifier (e.g., a BCG analog front end (AFE) 403), filter the amplified signal, and generate an analog voltage suitable for analog-to-digital conversion (ADC) by the processor.

FIG. 4 illustrates a block configuration of the sensor module 201 configured to measure ballistocardiogram (BCG) signals according to an embodiment.

Referring to FIG. 4, the sensor module 201 may include a processor 401, a piezoelectric element 402, a BCG analog front end (AFE) 403, an RGB indicator 404, a data storage unit 405, a communication unit 406, a motion sensor 407, an artificial intelligence (AI) model unit 408, a data processing unit 409, a noise removal unit 410, and a battery 411. However, the sensor module 201 is not limited to the components illustrated in FIG. 4, and certain components may be omitted or additional components may be provided. For example, the RGB indicator 404 may be omitted. As another example, the sensor module 201 may further include a pressure sensor configured to detect an air pressure inside the air tube 110 of the wearable device 101. The AI model unit 408 may include a first AI model 408-1 and a second AI model 408-2. The first AI model 408-1 may be used to process or pre-process air-pressure data or biometric signals acquired through the piezoelectric element 402. The second AI model 408-2 may be used to classify a symptom of a user based on processed data (e.g., heart rate variability (HRV) data) derived from output data of the first AI model 408-1 (e.g., BCG data).

According to one embodiment, although not illustrated in FIG. 4, the sensor module 201 may be operatively and/or electrically coupled to a driving unit configured to inject or discharge air into or from the air tube 110 of the wearable device 101. For example, the sensor module 201 may be disposed inside a housing of the driving unit.

According to one embodiment, although not illustrated in FIG. 4, the battery 411 may be operatively and/or electrically coupled to the driving unit. For example, the battery 411 may supply power to the driving unit. The battery 411 may be separate from the sensor module 201 and may supply electrical power to both the sensor module 201 and the driving unit.

According to one embodiment, although not illustrated in FIG. 4, the sensor module 201 may be located inside a silicone tube, and the sensor module 201 positioned inside the silicone tube may be disposed within the housing of the driving unit.

According to one embodiment, the processor 401 may be a dedicated processor used to control a specific system, such as a microcontroller unit (MCU). The processor 401 may be operatively and/or electrically coupled to the piezoelectric element 402, the BCG AFE 403, the RGB indicator 404, the data storage unit 405, the communication unit 406, the motion sensor 407, the AI model unit 408, the data processing unit 409, the noise removal unit 410, and the battery 411. The processor 401 may analyze and process a variety of data acquired from these components and may control the components based on the results of such analysis and processing.

According to one embodiment, the processor 401 may acquire air-pressure data inside the air tube 110 of the wearable device 101 through the piezoelectric element 402.

According to one embodiment, the BCG AFE 403 may be a circuit configured to amplify a small output voltage generated by the piezoelectric element 402, filter the amplified signal, and generate an analog voltage suitable for analog-to-digital conversion (ADC) by the processor 401.

According to one embodiment, the RGB indicator 404 may be a three-color LED that indicates an operation state of the processor 401.

According to one embodiment, the data storage unit 405 may store air-pressure data acquired through the piezoelectric element 402, output data generated by the AI model unit 408, and other related data.

According to one embodiment, the communication unit 406 may support data communication with an external device (e.g., the server 103). For example, the wearable device 101 may transmit biometric signals to the server 103 through the communication unit 406.

According to one embodiment, the motion sensor 407 may acquire movement data of a user wearing the wearable device 101. For example, the motion sensor 407 may include a three-axis motion sensor, an accelerometer, a geomagnetic sensor, and/or a gyroscope.

According to one embodiment, the motion sensor 407 may perform I²C communication with the processor 401. The motion sensor 407 may provide movement data obtained through the motion sensor 407 to the processor 401 through I²C communication.

According to one embodiment, the AI model unit 408 may include the first AI model 408-1 and the second AI model 408-2. The first AI model 408-1 may be an AI model used to process or pre-process air-pressure data or biometric signals (e.g., BCG signals) acquired through the piezoelectric element 402. That is, the first AI model 408-1 may perform raw data filtering using a template-matching technique to extract filtered data from processed data (e.g., data obtained through Fourier and wavelet transformations). The first AI model 408-1 may be referred to as a similarity-matching model. The raw data may include air-pressure data acquired through the piezoelectric element 402 or data pre-processed by the data processing unit 409. Pre-processing may include Fourier transform and wavelet transform.

According to one embodiment, the first AI model 408-1 may be trained such that, when raw data are input, the first AI model outputs filtered data using a set of normal BCG data (or normal biometric data) as training data. The normal BCG or biometric data sets may serve as template data. The filtered data may vary depending on the individual user based on how the model is trained. The normal biometric data sets may include at least one of a normal heart-rate data set, a normal HRV data set, a normal respiratory data set, a normal electrodermal activity data set, a normal PPG data set, a normal BVP data set, or a normal temperature data set.

According to one embodiment, the first AI model 408-1 may use, as input data, at least one of air-pressure data acquired through the piezoelectric element 402 or pre-processed data derived by the data processing unit 409, and may use normal BCG data (or normal biometric data) as output data for training.

According to one embodiment, the second AI model 408-2 may be an AI model used to classify user symptoms based on processed data (e.g., heart-rate variability (HRV) data) derived from output data of the first AI model 408-1 (e.g., BCG data).

According to one embodiment, the second AI model 408-2 may be trained such that, when processed data (e.g., HRV data) derived from output data of the first AI model 408-1 are input, the second AI model 408-2 outputs a type of symptom. The symptom may include a stress-related symptom, a respiration-related symptom, a pain-related symptom, a depression-related symptom, or a heart-condition-related symptom.

According to one embodiment, stress-related symptoms may reduce heart-rate variability. For example, anxiety, tension, irritability, and physical stress may lead to irregular heart-rate patterns and reduced HRV. The second AI model 408-2 may be trained using HRV data corresponding to such stress-related symptoms.

According to one embodiment, respiration-related symptoms, such as abnormal breathing patterns, airway obstruction, asthma, or dyspnea, may also reduce HRV. The second AI model 408-2 may be trained using HRV data corresponding to such conditions.

According to one embodiment, pain-related symptoms, such as chronic pain, neuralgia, or muscle pain, may cause irregular heart-rate patterns and HRV reduction. The second AI model 408-2 may be trained using HRV data corresponding to such conditions.

According to one embodiment, depression-related symptoms, such as low mood, fatigue, or lack of motivation, may also be associated with reduced HRV. The second AI model 408-2 may be trained using HRV data corresponding to such depression-related patterns.

According to one embodiment, heart-condition-related symptoms, such as heart failure, arrhythmia, or myocardial infarction, may reduce HRV variability. The second AI model 408-2 may be trained using HRV data corresponding to such conditions.

According to one embodiment, the data processing unit 409 may be a circuit configured to process sensor data (e.g., BCG raw data) acquired through the sensor module (e.g., the piezoelectric element 402) into input data for the AI model (e.g., the first AI model 408-1). For example, the data processing unit 409 may pre-process air-pressure data acquired through the piezoelectric element 402 to generate input data for the first AI model 408-1 of the AI model unit 408. The pre-processing may include Fourier transform and wavelet transform. The data processing unit 409 may apply a first Fourier transform to the air-pressure data, and then apply a wavelet transform to the Fourier-transformed data to generate transformed datasets. Data obtained through the Fourier transform may be first transformed data, and data obtained through the wavelet transform may be second transformed data.

According to one embodiment, the noise removal unit 410 may remove noise included in sensing data detected through the piezoelectric element 402 based on movement data acquired through the motion sensor 407 (e.g., a three-axis motion sensor, accelerometer, geomagnetic sensor, and/or gyroscope). For example, the noise removal unit 410 may remove noise by superimposing motion signals (e.g., x, y, z axis accelerometer signals) onto sensing data (e.g., air-pressure data) and removing amplified signals corresponding to noise.

According to one embodiment, the battery 411 may supply electrical power to operate the sensor module 201. The battery 411 may support wired and/or wireless charging. When the battery 411 is in a charging state, the communication unit 406 may be activated. For example, in response to determining that the battery 411 is in a charging state, the processor 401 may activate the communication unit 406 to perform data communication with an external device (e.g., the server 103). That is, when the battery 411 is in a charging state, the processor 401 may transmit sensing data to the server 103 through the communication unit 406. The sensing data may include sensing data detected through the piezoelectric element 402 (e.g., air-pressure data) or processed data (e.g., data pre-processed by the data processing unit 409 including Fourier and wavelet transforms, and output data generated through the first AI model 408-1).

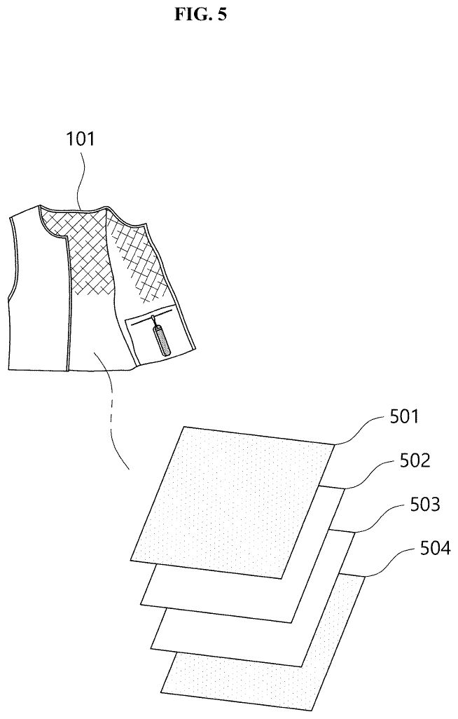

FIG. 5 illustrates a sectional view of a fabric structure of the wearable device 101 according to an embodiment.

Referring to FIG. 5, the front portion and the rear portion of the wearable device 101 may be formed of a fabric structure having four layers. That is, the layers of the wearable device 101 may be stacked from top to bottom in the order of a first layer 501, a second layer 502, a third layer 503, and a fourth layer 504. The layers of the wearable device 101 may include the first layer 501 formed of a fabric material, the second layer 502 formed of a urethane film, the third layer 503 formed of a urethane film, and the fourth layer 504 formed of a fabric material.

The urethane film of the second layer 502 and the urethane film of the third layer 503 may be bonded to each other through a high-frequency welding process. The fabric material of the first layer 501 and the urethane film of the second layer 502 may be bonded to each other through a binding process. The urethane film of the third layer 503 and the fabric material of the fourth layer 504 may also be bonded to each other through a binding process.

According to one embodiment, a process for forming the four layers constituting the fabric of the wearable device 101 may include a first process of generating a first composite by bonding the first layer 501 and the second layer 502 through a binding process, and generating a second composite by bonding the third layer 503 and the fourth layer 504 through a binding process; and a second process of bonding the first composite and the second composite to each other through a high-frequency welding method. In the second process, the urethane-film surfaces of the first composite and the second composite may be bonded to each other.

According to one embodiment, the fabric material may include cotton, polyester, rayon, nylon, wool, cashmere, silk, linen, or spandex.

According to one embodiment, the urethane film may refer to a thin film made of polyurethane synthetic fiber. Polyurethane may be durable, flexible, and elastically strong, and may have a transparent property.

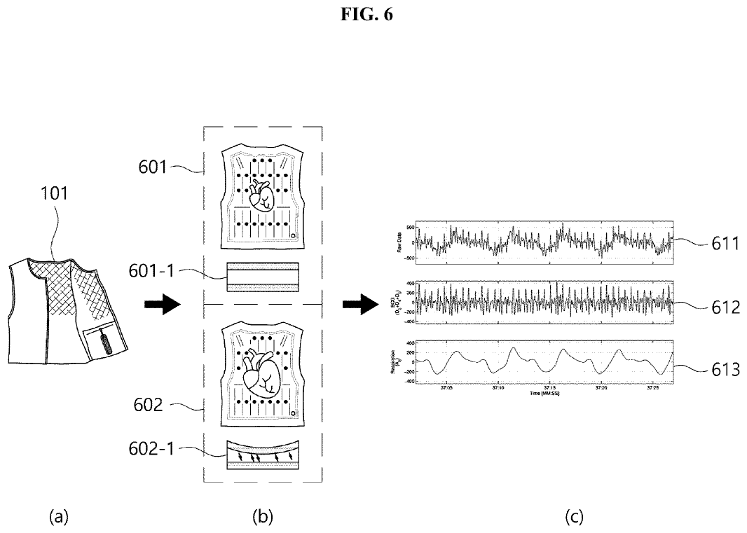

FIG. 6 illustrates a conceptual diagram of a technique for acquiring biometric information of a user using the sensor module 201 provided in the wearable device 101 according to an embodiment.

Referring to FIG. 6, FIG. 6(a) illustrates the wearable device 101 equipped with a sensor (e.g., the sensor module 201) configured to acquire biometric information of a user in real time when the user wears the wearable device 101. FIG. 6(b) illustrates contraction and expansion of a blood vessel of the user according to a biometric condition (e.g., cardiac activity or respiratory activity) when the user wears the wearable device 101. FIG. 6(c) illustrates a graph representing biometric information of the user wearing the wearable device 101, acquired in real time through the sensor of the wearable device 101.

According to one embodiment, when a user wearing the wearable device 101 exhales or when the user's heart contracts (601), the user's blood vessels may remain in a normal state or expand. When the user wearing the wearable device 101 inhales or when the user's heart expands (602), the user's blood vessels may contract relative to their normal state.

According to one embodiment, the wearable device 101 may acquire biometric information of the user through the sensor module 201. The wearable device 101 may acquire raw data 611 representing biometric information of the user through the sensor module 201. The wearable device 101 may generate BCG data 612 and respiration data 613 by processing the raw data through the data processing unit 409. For example, the wearable device 101 may apply a Fourier transform and a wavelet transform to the raw data through the data processing unit 409. The wearable device 101 may input the transformed data obtained through the Fourier and wavelet transformations into the first AI model 408-1 to generate filtered data. The filtered data may include BCG data 612 or respiration data 613.

According to one embodiment, the Fourier transform may refer to a process of separating a frequency corresponding to a biological rhythm (e.g., a heart rhythm or a respiratory rhythm) from raw data measured through the piezoelectric element 402.

According to one embodiment, the wavelet transform may refer to a process of decomposing a signal into various frequency bands by stretching or compressing the length of the signal along the time axis, and a process of calculating correlation coefficients between the raw data and a wavelet function modeled according to a specific rule while varying the time scale of the wavelet function.

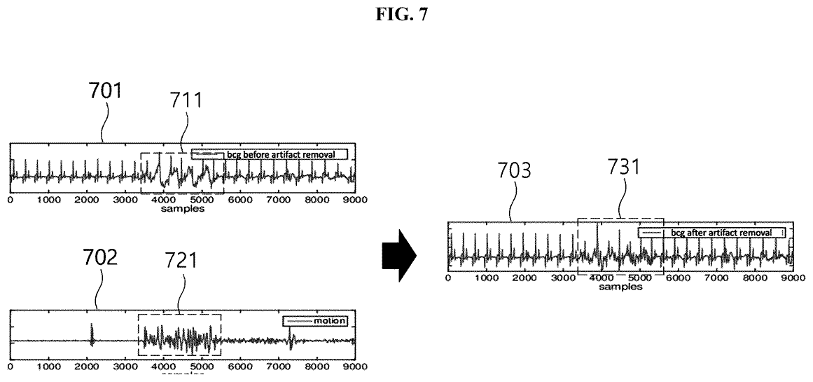

FIG. 7 illustrates a conceptual diagram of a technique for correcting biometric information using movement information of a user according to an embodiment.

Referring to FIG. 7, the wearable device 101 may remove noise included in air-pressure data acquired through the piezoelectric element 402 by using the noise removal unit 410. For example, the wearable device 101 may pre-process raw data (e.g., air-pressure data) acquired through the piezoelectric element 402 of the sensor module 201 and may acquire biometric data (e.g., BCG data and respiration data) through the pre-processing. The biometric data obtained through the pre-processing may include noise generated due to movements of the user. For example, the graph 701 in FIG. 7(a) illustrates biometric data obtained through the pre-processing, and noise may be included in a first portion 711. The graph 702 in FIG. 7(a) illustrates movement data of the user acquired through the motion sensor 407, and movement data generated due to user motion may be included in a second portion 721.

According to one embodiment, the graph 701 in FIG. 7(b) may represent biometric data from which noise has been removed by using the movement data shown in the graph 702. For example, because of the movement data shown in the second portion 721 of the graph 702, the noise shown in the first portion 711 of the graph 701 may be removed by superimposing the biometric data of the graph 701 and the movement data of the graph 702 and eliminating amplified signals, thereby generating noise-reduced data as shown in a third portion 731 of the graph 703.

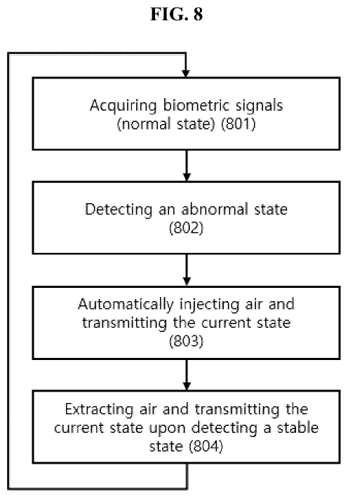

FIG. 8 illustrates a flowchart of a method for caring for a psychological state of a user based on biometric information using the wearable device 101 according to an embodiment.

The operations of the wearable device 101 described below may be performed in a different order or simultaneously.

In operation 801, the wearable device 101 may acquire biometric information of a user wearing the wearable device 101 in real time through the sensor module 201. For example, the wearable device 101 may detect an air pressure inside the air tube 110 of the wearable device 101 through the piezoelectric element 402 of the sensor module 201 and may acquire air-pressure data. The air pressure may vary depending on a cardiac rhythm or a respiratory condition of the user.

According to one embodiment, the wearable device 101 may process the air-pressure data into biometric data through the data processing unit 409 and the AI model unit 408. The air-pressure data may be understood as raw data or BCG data. The wearable device 101 may process the raw data through the data processing unit 409. For example, the wearable device 101 may apply a first Fourier transform to the raw data and may apply a second wavelet transform thereafter.

According to one embodiment, by applying the Fourier transform and the wavelet transform to the raw data, the wearable device 101 may acquire BCG data and respiration data. The BCG data and the respiration data may correspond to second transformed data.

According to one embodiment, to obtain BCG data (or the second transformed data) from the raw data, the wearable device 101 may perform a Fourier transform by separating a first frequency corresponding to a cardiac rhythm. The wearable device 101 may then perform a wavelet transform on the first transformed data using a wavelet function modeled according to the cardiac rhythm.

According to one embodiment, to obtain respiration data (or the second transformed data) from the raw data, the wearable device 101 may perform a Fourier transform by separating a second frequency corresponding to a respiratory rhythm. The wearable device 101 may then perform a wavelet transform on the respiration-related transformed data using a wavelet function modeled according to the respiratory rhythm.

According to one embodiment, the wearable device 101 may input the second transformed data (generated through the Fourier and wavelet transforms) into the first AI model 408-1 of the AI model unit 408, and may acquire biometric data in real time based on output data of the first AI model. The biometric data may refer to data processed using the template-matching technique of the first AI model, also referred to as raw-data filtered data. The biometric data may include filtered BCG data or filtered respiration data.

In operation 802, the wearable device 101 may detect an abnormal state of the user based on the biometric data (e.g., BCG data or respiration data) or processed biometric data (e.g., heart-rate data or heart-rate-variability data) acquired in real time. For example, the wearable device 101 may detect an abnormal state by determining whether a value of the biometric data exceeds a predetermined range.

According to one embodiment, the wearable device 101 may detect an abnormal state of the user based on whether heart-rate data exceed a predetermined range. The predetermined range may include a first value and a second value corresponding to a normal heart-rate interval by age. For example, the average resting heart rate of an adult aged 20 or older is typically about 70 to 75 beats per minute; thus, the first value may be 70 and the second value may be 75. However, these values are merely exemplary. When the heart rate exceeds the predetermined range, the wearable device 101 may determine that the user is anxious or under stress.

According to one embodiment, the wearable device 101 may detect an abnormal state based on whether heart-rate-variability (HRV) data exceed a predetermined range. The predetermined range may include a third value and a fourth value corresponding to a normal HRV interval by age. For example, when psychological stability is high or stress is low, HRV tends to be high, and the difference between the third and fourth values may be a first difference. When a psychological state is unstable or stress is elevated, HRV tends to decrease, and the difference between the third and fourth values may be a second difference. The first difference may be greater than the second difference. When HRV exceeds or deviates from the predetermined range, the wearable device 101 may determine that the user is anxious or experiencing stress.

According to one embodiment, the wearable device 101 may detect an abnormal state based on whether respiration data exceed a predetermined range. The predetermined range may include a fifth value and a sixth value corresponding to a normal respiration rate, e.g., 12 breaths per minute to 20 breaths per minute. When the respiration rate exceeds this range, the wearable device 101 may determine that the user is anxious or under stress.

In operation 803, when the wearable device 101 detects that the user is anxious or stressed—that is, when the user is in an abnormal state—the wearable device 101 may automatically control the driving unit to inject air into the air tube 110. The wearable device 101 may transmit, to an external device (e.g., the server 103 or a user terminal), at least one of state information indicating the abnormal state or information related to controlling the driving unit.

According to one embodiment, the wearable device 101 may determine at least one of a type of psychological state, a stress level, or a type of symptom of the user based on the acquired biometric information.

A type of psychological state may include happiness, sadness, comfort, anxiety, anger, or the like. A stress level may be divided into stages. For example, stress levels may be classified from a first level to a fifth level, where higher levels correspond to higher stress. A type of symptom may include a stress-related symptom, a respiration-related symptom, a pain-related symptom, a depression-related symptom, or a heart-condition-related symptom.

According to one embodiment, the wearable device 101 may select an air-injection method corresponding to the detected psychological state, stress level, and symptom type. The air-injection method may include at least one of an air-injection intensity, an air-injection speed, an air-injection hold time, or a region of the body to which pressure is applied. Based on the selected air-injection method, the wearable device 101 may control the driving unit to inject air into the air tube 110.

For example, when the user is determined to be anxious, the wearable device 101 may inject air into the air tube 110 at a first pressure and a first speed and maintain the airflow for a first duration. As another example, when the user is determined to be angry, the wearable device 101 may inject air at a second pressure greater than the first pressure and a second speed greater than the first speed, and may maintain air injection for a second duration longer than the first duration. These air-injection methods are merely illustrative.

In operation 804, the wearable device 101 may determine that the psychological state of the user has stabilized based on biometric information acquired in real time. For example, when a value of the biometric data falls within a predetermined range, the wearable device 101 may determine that the user's psychological state is stable. For example, when at least one of BCG data, heart-rate data, HRV data, or respiration data falls within the predetermined range, the user may be determined to be in a stable psychological state.

According to one embodiment, when the psychological state of the user is determined to be stable, the wearable device 101 may control the driving unit to discharge air from the air tube 110. The wearable device 101 may transmit, to an external device (e.g., the server 103 or a user terminal), at least one of state information indicating that the user is in a stable state or information related to controlling the driving unit.

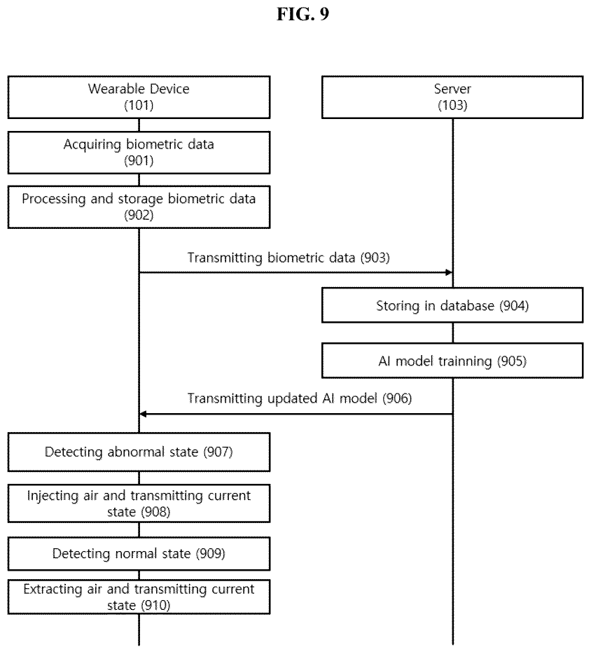

FIG. 9 illustrates a flowchart of operations between the wearable device 101 and the server 103 for caring for a psychological state using biometric information according to an embodiment.

The operations of the wearable device 101 and the server 103 described below may be performed in a different order or simultaneously.

In operation 901, the wearable device 101 may acquire biometric data through a sensor (e.g., the sensor module 201) provided in the wearable device 101. The biometric data may include at least one of BCG data, heart-rate data, heart-rate-variability data, respiration data, electrodermal-activity data, photoplethysmography (PPG) data, blood-volume-pulse (BVP) data, or thermal (temperature) data. The biometric data may refer to data obtained by processing raw data (or pressure data or air-pressure data) detected by the piezoelectric element 402 of the sensor module 201 of the wearable device 101.

According to one embodiment, the wearable device 101 may acquire air-pressure data (or pressure data or raw data) representing an air pressure inside the air tube 110 of the wearable device 101 through the piezoelectric element 402 of the sensor module 201. The wearable device 101 may process and handle the air-pressure data through the data processing unit 409 and the AI model unit 408 to obtain biometric data.

In operation 902, the wearable device 101 may store the biometric data in the data storage unit 405. The wearable device 101 may perform an operation of removing noise included in the biometric data through the noise removal unit 410. According to one embodiment, the wearable device 101 may remove noise included in sensing data (e.g., pressure data, air-pressure data) detected through the piezoelectric element 402 by using the noise removal unit 410. For example, the wearable device 101 may remove noise from the sensing data by superimposing the sensing data with movement data detected through the motion sensor 407 and removing amplified signals. The movement data may include x, y, and z axis data of an accelerometer.

In operation 903, the wearable device 101 may transmit the biometric data to the server 103 through the communication unit 406. Although not illustrated in FIG. 9, the wearable device 101 may transmit, in addition to the biometric data, at least one of raw data, pre-processed data based on the raw data (e.g., first transformed data, second transformed data), or filtered data processed using the first AI model to the server 103. The raw data may refer to pressure data or air-pressure data. To transmit the biometric data to the server 103 through the communication unit 406, the sensor module 201 or the driving unit of the wearable device 101 may be assumed to be in a charging state. For example, only when the sensor module 201 or the driving unit of the wearable device 101 is in a charging state may the wearable device 101 perform data communication with the server 103 through the communication unit 406.

According to one embodiment, the wearable device 101 may also transmit personal information of the user (e.g., age, gender, height, weight, degree of disability, etc.) to the server 103 through the communication unit 406.

In operation 904, the server 103 may store the biometric data and personal information received from the wearable device 101 in a database. The server 103 may receive the biometric data and personal information from the wearable device 101 only when the server 103 is wirelessly connected to the wearable device 101.

In operation 905, the server 103 may train an artificial intelligence model using the received biometric data and personal information. The AI model may be substantially identical to the AI models (e.g., the first AI model 408-1 and the second AI model 408-2) executed in the wearable device 101.

According to one embodiment, the server 103 may train and execute a third AI model configured to perform a raw-data filtering operation to extract filtered data from processed data (e.g., data obtained through Fourier and wavelet transforms) using a template-matching technique. The third AI model may be trained and executed in substantially the same manner as the first AI model 408-1. The third AI model may be referred to as a signal-matching model that uses a template-matching technique. For example, the third AI model may be trained using normal BCG data sets (or normal biometric data sets) such that, when raw data are input, the third AI model outputs filtered data.

According to one embodiment, the server 103 may extract features from the raw data received from the wearable device 101, perform vector quantization, and generate templates (or template data) for use in template matching.

According to one embodiment, the server 103 may train and execute a fourth AI model configured to determine a psychological state, a stress level, and a type of symptom of the user based on the user's personal information and biometric information. The fourth AI model may be trained and executed in substantially the same manner as the second AI model 408-2. The fourth AI model may be referred to as a personalized learning model used to classify user symptoms. For example, the fourth AI model may be trained such that, when processed data (e.g., HRV data or respiration data) derived from output data (e.g., BCG data) of the third AI model are input, the fourth AI model outputs a type of symptom.

In operation 906, the server 103 may transmit the generated templates (or template data) to the wearable device 101. The server 103 may update AI models (e.g., the third AI model and the fourth AI model) trained using data (e.g., raw data, biometric data, etc.) received from the wearable device 101 and may transmit the updated AI models to the wearable device 101.

In operation 907, operation 907 may be substantially identical to operation 802. The wearable device 101 may detect an abnormal state of the user by using the updated AI models received from the server 103. The wearable device 101 may input pre-processed raw data (e.g., second transformed data) into the updated first AI model 408-1, output filtered data, process the filtered data, and input the processed data into the updated second AI model 408-2. The wearable device 101 may determine whether the state of the user is abnormal based on the output of the updated second AI model.

In operation 908, operation 908 may be substantially identical to operation 803. In response to detecting that the user is in an abnormal state, the wearable device 101 may automatically inject air into the air tube 110 and may transmit a current state of the user to an external device (e.g., the server 103 or a user terminal).

In operation 909, operation 909 may be substantially identical to the stable-state detection operation of operation 804. The wearable device 101 may determine that the state of the user is stable when a value of the biometric information falls within a predetermined range.

In operation 910, operation 910 may be substantially identical to the air-discharge and current-state transmission operation of operation 804. When the user is determined to be in a stable state, the wearable device 101 may discharge air from the air tube 110 and may transmit at least one of information indicating that the user is in a stable state or information indicating that the driving unit has been controlled to discharge air from the air tube 110 to an external device (e.g., the server 103 or a user terminal).

According to one embodiment, a wearable device for caring for a psychological state of a user based on biometric information may include an outer shell including a front portion positioned at a chest region of the user and a rear portion positioned at a back region of the user. The outer shell may include a first surface facing a body of the user and a second surface facing away from the body of the user. The wearable device may further include an air tube disposed between the first surface and the second surface, an air inlet provided at one end of the air tube such that air is injected into or discharged from the air tube, a driving unit configured to inject air into or discharge air from the air tube through the air inlet, a silicone tube extending from the driving unit and connected to the air inlet, a sensor module disposed inside the silicone tube and including a piezoelectric element configured to sense pressure, and a processor disposed inside the silicone tube and operatively coupled to the driving unit and the sensor module.

According to one embodiment, the processor may be configured to acquire air-pressure data representing an air pressure inside the air tube through the piezoelectric element of the sensor module, determine a state of the user based on the air-pressure data, the state of the user including at least one of a psychological state, a stress level, or a type of physical symptom, and control the driving unit to inject air into the air tube based on the determined state of the user.

According to one embodiment, the wearable device may further include a data processing unit configured to process data acquired through the piezoelectric element. The processor may be configured to apply a first Fourier transform to the air-pressure data through the data processing unit, the first Fourier transform being a transform configured to separate a first frequency corresponding to a cardiac rhythm from the air-pressure data. The processor may be configured to apply a second Fourier transform to the air-pressure data through the data processing unit, the second Fourier transform being a transform configured to separate a second frequency corresponding to a respiratory rhythm from the air-pressure data, and may acquire BCG data and respiration data of the user from the air-pressure data based on the first and second Fourier transforms.

According to one embodiment, the processor may be configured to perform a first wavelet transform on air-pressure data to which the first Fourier transform has been applied by using a first wavelet function modeled according to the cardiac rhythm through the data processing unit, perform a second wavelet transform on air-pressure data to which the second Fourier transform has been applied by using a second wavelet function modeled according to the respiratory rhythm through the data processing unit, and acquire the BCG data and the respiration data of the user from the air-pressure data based on the first and second wavelet transforms.

According to one embodiment, the wearable device may further include an AI model unit configured to operate a first AI model trained to extract filtered data from the BCG data and the respiration data derived from the first and second wavelet transforms using a template-matching technique. The processor may be configured to input the BCG data and the respiration data into the first AI model of the AI model unit to acquire filtered BCG data and filtered respiration data. The first AI model may be trained, using normal BCG data sets and normal respiration data sets as training data, to output the filtered BCG data and the filtered respiration data when the BCG data and the respiration data are input.

According to one embodiment, the AI model unit may further include a second AI model trained to determine the state of the user based on the filtered BCG data and the filtered respiration data. The processor may be configured to input the filtered BCG data and the filtered respiration data into the second AI model to determine the state of the user and to control the driving unit to inject air into the air tube based on the determined state of the user. A type of physical symptom included in the state of the user may include at least one of a stress-related symptom, a respiration-related symptom, a pain-related symptom, a depression-related symptom, or a heart-condition-related symptom. The second AI model may be trained to output the state of the user when biometric data of the user are input, using biometric data corresponding to states of the user as training data.

According to one embodiment, the wearable device may further include a motion sensor. The processor may be configured to acquire movement data of the user through the motion sensor and remove noise included in the filtered BCG data and the filtered respiration data based on the movement data.

According to one embodiment, the wearable device may further include a motion sensor. The processor may be configured to acquire movement data of the user through the motion sensor and remove noise included in the air-pressure data based on the movement data.

According to one embodiment, the first surface and the second surface may be formed of a fabric material, a first urethane film may be disposed between the first surface and the second surface, and a second urethane film may be disposed between the first urethane film and the second surface. The first surface and the first urethane film may be bonded to each other through binding, the second surface and the second urethane film may be bonded to each other through binding, and the first urethane film and the second urethane film may be bonded to each other through high-frequency welding.

According to one embodiment, the fabric material may include at least one of cotton, polyester, rayon, nylon, wool, cashmere, silk, linen, or spandex.

According to one embodiment, the wearable device may further include a battery and a communication unit. The processor may be configured to activate the communication unit only while the battery is in a charging state and transmit the air-pressure data to an external server through the communication unit.

What is claimed is:

1. A wearable device for caring for a psychological state of a user based on biometric information of the user, the wearable device comprising:
an outer shell including a front portion positioned at a chest region of the user and a back portion positioned at a back region of the user, the outer shell comprising a first surface facing the user's body and a second surface facing away from the user's body;
an air tube disposed between the first surface and the second surface;
an air inlet provided at one end of the air tube, the air inlet allowing air to be injected into or discharged from the air tube;
a driving unit configured to inject air into or discharge air from the air tube through the air inlet;
a silicone tube extending from the driving unit and connected to the air inlet, a first end of the silicone tube forming an opening and being connected to the air inlet, and a second end of the silicone tube being formed in a sealed structure;
a sensor module comprising a piezoelectric element configured to sense pressure, the piezoelectric element being disposed inside the silicone tube at a predetermined distance from the sealed second end of the silicone tube; and
a processor disposed inside the silicone tube and operatively coupled to the driving unit and the sensor module,
wherein the processor is configured to:
acquire air-pressure data representing an internal air pressure of the air tube through the piezoelectric element of the sensor module;
determine a state of the user based on the air-pressure data; and
control the driving unit to inject air into the air tube based on the determined state of the user.

2. The wearable device of claim 1,
wherein the state of the user comprises at least one of a psychological state, a stress level, or a physical symptom.

3. The wearable device of claim 1, further comprising a data processing unit configured to process data acquired through the piezoelectric element,
wherein the processor is configured to:
apply a first Fourier transform to the air-pressure data through the data processing unit;
apply a second Fourier transform to the air-pressure data through the data processing unit; and
acquire ballistocardiogram (BCG) data and respiration data of the user from the air-pressure data based on the first and second Fourier transforms.

4. The wearable device of claim 3,
wherein the first Fourier transform separates a first frequency corresponding to a cardiac rhythm from the air-pressure data, and
wherein the second Fourier transform separates a second frequency corresponding to a respiratory rhythm from the air-pressure data.

5. The wearable device of claim 4,
wherein the processor is further configured to:
perform a first wavelet transform on the air-pressure data to which the first Fourier transform is applied, using a first wavelet function modeled based on the cardiac rhythm;
perform a second wavelet transform on the air-pressure data to which the second Fourier transform is applied, using a second wavelet function modeled based on the respiratory rhythm; and
acquire the ballistocardiogram data and the respiration data of the user from the air-pressure data based on the first and second wavelet transforms.

6. The wearable device of claim 5, further comprising an artificial intelligence (AI) model unit configured to execute a first AI model trained to extract filtered data from the ballistocardiogram data and the respiration data subjected to the first and second wavelet transforms using a template-matching technique,
wherein the processor is configured to:
input the ballistocardiogram data and the respiration data into the first AI model of the AI model unit, and acquire filtered ballistocardiogram data and filtered respiration data; and
wherein the first AI model is trained, using a normal ballistocardiogram dataset and a normal respiration dataset as training data, to output the filtered ballistocardiogram data and the filtered respiration data when the ballistocardiogram data and the respiration data are input.

7. The wearable device of claim 6,
wherein the AI model unit further comprises a second AI model trained to determine the state of the user based on the filtered ballistocardiogram data and the filtered respiration data,
and wherein the processor is configured to:
input the filtered ballistocardiogram data and the filtered respiration data into the second AI model to determine the state of the user; and
control the driving unit to inject air into the air tube based on the determined state of the user,
wherein the physical symptoms included in the state of the user comprise at least one of a stress-related symptom, a respiration-related symptom, a pain-related symptom, a depression-related symptom, or a cardiac-disease-related symptom, and
wherein the second AI model is trained to output the state of the user when biometric data corresponding to the user's state are input.

8. The wearable device of claim 6, further comprising a motion sensor,
wherein the processor is configured to:
acquire motion data of the user through the motion sensor; and
remove noise included in the filtered ballistocardiogram data and the filtered respiration data based on the motion data.

9. The wearable device of claim 1, further comprising a motion sensor,
wherein the processor is configured to:
acquire motion data of the user through the motion sensor; and
remove noise included in the air-pressure data based on the motion data.

10. The wearable device of claim 1,
wherein the first surface and the second surface are formed of a fabric material,
wherein a first urethane film is disposed between the first surface and the second surface, and a second urethane film is disposed between the first urethane film and the second surface,
wherein the first surface and the first urethane film are bonded to each other through binding,
wherein the second surface and the second urethane film are bonded to each other through binding, and
wherein the first and second urethane films are bonded to each other through high-frequency welding.

11. The wearable device of claim 10,
wherein the fabric material comprises at least one of cotton, polyester, rayon, nylon, wool, cashmere, silk, linen, or spandex.

12. The wearable device of claim 1, further comprising:
a battery; and
a communication unit,
wherein the processor is configured to:
activate the communication unit only when the battery is in a charging state; and
transmit the air-pressure data to an external server through the communication unit.
